(19) United States
(12) Patent Application Publication
(10) Pub. No.: US 2024/0299081 A1
(43) Pub. Date: Sep. 12, 2024

(54) KNIFE ASSEMBLIES FOR USE WITH SURGICAL INSTRUMENTS AND SYSTEMS

(71) Applicant: Covidien LP, Mansfield, MA (US)

(72) Inventors: Allen E. Honegger, Berthoud, CO (US); James J. Crawford, Lyons, CO (US)

(21) Appl. No.: 18/667,013

(22) Filed: May 17, 2024

Related U.S. Application Data
(63) Continuation of application No. 16/702,762, filed on Dec. 4, 2019.
(60) Provisional application No. 62/776,268, filed on Dec. 6, 2018.

(51) Int. Cl. A61B 18/14; A61B 17/00; A61B 18/00; A61B 18/12; A61B 18/18; A61B 34/00; A61B 34/35

(52) U.S. Cl. CPC A61B 18/1445; A61B 2017/00199; A61B 2017/00526; A61B 2017/00734; A61B 2017/00867; A61B 2018/00077; A61B 2018/00589; A61B 2018/00595; A61B 2018/0063; A61B 2018/00922; A61B 18/1206; A61B 2018/1455; A61B 2018/1807; A61B 2018/1823; A61B 2034/256; A61B 34/35

(57) ABSTRACT

An electrosurgical instrument includes first and second jaw members each including an outer jaw housing, a tissue-treating plate, and a longitudinally-extending knife channel. The electrosurgical instrument also includes a knife actuator and a knife assembly operably coupled to the knife actuator. The knife assembly includes an elongated tube defining a longitudinal lumen and an elongated shaft having a proximal portion coupled to the knife actuator and a distal portion configured to be received through the longitudinal lumen. The distal portion of the elongated shaft includes an enlarged distal end portion configured to prevent movement of the elongated shaft relative to the elongated tube. The knife assembly also includes a knife blade having a pair of opposing lateral sides and a sharpened distal end configured to cut tissue. The elongated tube is configured to be coupled to one of the pair of opposing lateral sides of the knife blade.

### CROSS-REFERENCE TO RELATED APPLICATIONS

This application is a continuation of U.S. patent application Ser. No. 16/702,762, filed on Dec. 4, 2019, which claims the benefit of the filing date of provisional U.S. Patent Application No. 62/776,268, filed on Dec. 6, 2018.

### FIELD

The present disclosure relates to surgical devices and, more particularly, to knife assemblies for use with surgical instruments and systems.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife or cutting member utilized to effectively sever the treated tissue.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

In accordance with aspects of the present disclosure, an electrosurgical instrument is provided. The electrosurgical instrument includes first and second jaw members each including an outer jaw housing, a tissue-treating plate, and a longitudinally-extending knife channel defined therethrough. At least one of the first or second jaw members is pivotable relative to the other between a spaced-apart position and an approximated position. The electrosurgical instrument also includes a knife actuator and a knife assembly operably coupled to the knife actuator. The knife actuator is configured to advance at least a portion of the knife assembly through the knife channel to cut tissue disposed between the jaw members when the jaw members are in the approximated position. The knife assembly includes an elongated tube defining a longitudinal lumen therethrough and an elongated shaft having a proximal portion coupled to the knife actuator and a distal portion configured to be received through the longitudinal lumen defined by the elongated tube. The distal portion of the elongated shaft includes an enlarged distal end portion configured to abut the elongated tube to prevent movement of the elongated shaft relative to the elongated tube. The knife assembly also includes a knife blade having a pair of opposing lateral sides and a sharpened distal end configured to cut tissue. The elongated tube is configured to be coupled to one of the pair of opposing lateral sides of the knife blade to couple the knife blade to the distal portion of the elongated shaft such that longitudinal movement of the elongated shaft effects corresponding longitudinal movement of the knife blade.

In an aspect of the present disclosure, the elongated tube and the knife blade are formed of a first material and the elongated shaft is formed of a second material different from the first material.

In another aspect of the present disclosure, the elongated tube and the knife blade are formed of stainless steel.

In another aspect of the present disclosure, the elongated tube is formed of stainless steel and the elongated shaft is formed of Nitinol.

In another aspect of the present disclosure, the elongated tube and the knife blade are formed of stainless steel and the elongated shaft is formed of Nitinol.

In yet another aspect of the present disclosure, a shaft stop is disposed on one of the pair of opposing lateral sides of the knife blade and is configured to prevent movement of the elongated shaft relative to the knife blade.

In another aspect of the present disclosure, a diameter of the enlarged distal end portion of the elongated shaft is larger than a diameter of the longitudinal lumen defined through the elongated tube.

In yet another aspect of the present disclosure, the elongated tube is welded to one of the pair of opposing lateral sides of the knife blade.

In another aspect of the present disclosure, the enlarged distal end portion is crimped to the elongated tube.

In accordance with aspects of the present disclosure, a knife assembly for use with a surgical instrument or surgical system is provided. The knife assembly includes an elongated tube defining a longitudinal lumen therethrough and an elongated shaft having a proximal portion configured to be coupled to a knife actuator for effecting longitudinal movement of the elongated shaft and a distal portion configured to be received through the longitudinal lumen defined by the elongated tube. The distal portion of the elongated shaft includes an enlarged distal end portion configured to abut the elongated tube to prevent movement of the elongated shaft relative to the elongated tube. The knife assembly also includes a knife blade having a pair of opposing lateral sides and a sharpened distal end configured to cut tissue. The elongated tube is configured to be coupled to one of the pair of opposing lateral sides of the knife blade to couple the knife blade to the distal portion of the elongated shaft.

In an aspect of the present disclosure, the elongated tube and the knife blade are formed of a first material and the elongated shaft is formed of a second material different from the first material.

In another aspect of the present disclosure, the elongated tube and the knife blade are formed of stainless steel.

In another aspect of the present disclosure, the elongated tube is formed of stainless steel and the elongated shaft is formed of Nitinol.

In another aspect of the present disclosure, the elongated tube and the knife blade are formed of stainless steel and the elongated shaft is formed of Nitinol.

In yet another aspect of the present disclosure, a shaft stop is disposed on one of the pair of opposing lateral sides of the knife blade and is configured to prevent movement of the elongated shaft relative to the knife blade.

In another aspect of the present disclosure, a diameter of the enlarged distal end portion of the elongated shaft is larger than a diameter of the longitudinal lumen defined through the elongated tube.

In yet another aspect of the present disclosure, the elongated tube is welded to one of the pair of opposing lateral sides of the knife blade.

In accordance with aspects of the present disclosure, a method of manufacturing a knife assembly for use with a surgical instrument or surgical system to cut tissue is provided. The method includes coupling an elongated tube to one of a pair of opposing lateral sides of a knife blade, inserting an elongated shaft through a longitudinal lumen defined by the elongated tube, and heating a distal end portion of the elongated shaft to distort the distal end portion to prevent movement of the elongated shaft to the knife blade.

In an aspect of the present disclosure, the method also includes heating the distal end portion of the elongated shaft prior to inserting the elongated shaft through the longitudinal lumen.

In another aspect of the present disclosure, the method also includes heating the distal end portion of the elongated shaft subsequent to inserting the elongated shaft through the longitudinal lumen.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements and:

FIG. 1A is a perspective view of endoscopic surgical forceps exemplifying the aspects and features of the present disclosure, wherein the jaw members of the endoscopic surgical forceps are disposed in a spaced-apart position;

FIG. 1B is an enlarged perspective view of the endoscopic surgical forceps of FIG. 1A, wherein the jaw members of the endoscopic surgical forceps are disposed in a spaced-apart position;

FIG. 1C is an enlarged perspective view of the endoscopic surgical forceps of FIG. 1A, wherein the jaw members of the endoscopic surgical forceps are disposed in an approximated position;

FIG. 2 is a perspective view of an open surgical forceps exemplifying the aspects and features of the present disclosure;

FIG. 3 is a schematic illustration of a robotic surgical system exemplifying the aspects and features of the present disclosure;

FIG. 4A is an exploded perspective view of another knife assembly configured for use with the forceps of FIGS. 1A-1C, the forceps of FIG. 2, the system of FIG. 3, or any other suitable surgical instrument in accordance with embodiments of the present disclosure;

FIG. 4B is an assembled perspective view of the knife assembly of FIG. 4A;

FIG. 4C is a cross-sectional view taken along line 4C-4C of FIG. 4B;

FIG. 5A is an exploded perspective view of another knife assembly configured for use with the forceps of FIGS. 1A-IC, the forceps of FIG. 2, the system of FIG. 3, or any other suitable surgical instrument in accordance with embodiments of the present disclosure;

FIG. 5B is an assembled perspective view of the knife assembly of FIG. 5A;

FIG. 5C is a cross-sectional view taken along line 5C-5C of FIG. 5B;

FIGS. 6A and 6B are top perspective views of respective knife assemblies configured for use with the forceps of FIGS. 1A-IC, the forceps of FIG. 2, the system of FIG. 3, or any other suitable surgical instrument in accordance with embodiments of the present disclosure;

FIG. 6C is a side view of a distal portion of the knife assembly of FIGS. 6A and 6B illustrating the coupling between the elongated tube and the knife blade;

FIG. 7A is a partially exploded perspective view of another knife assembly configured for use with the forceps of FIGS. 1A-1C, the forceps of FIG. 2, the system of FIG. 3, or any other suitable surgical instrument in accordance with embodiments of the present disclosure;

FIG. 7B is an assembled perspective view of the knife assembly of FIG. 7A; and

FIG. 7C is a side view of the knife assembly of FIGS. 7A and 7B showing a shaft stop coupled to the knife blade in accordance with some embodiments of the present disclosure.

### DETAILED DESCRIPTION

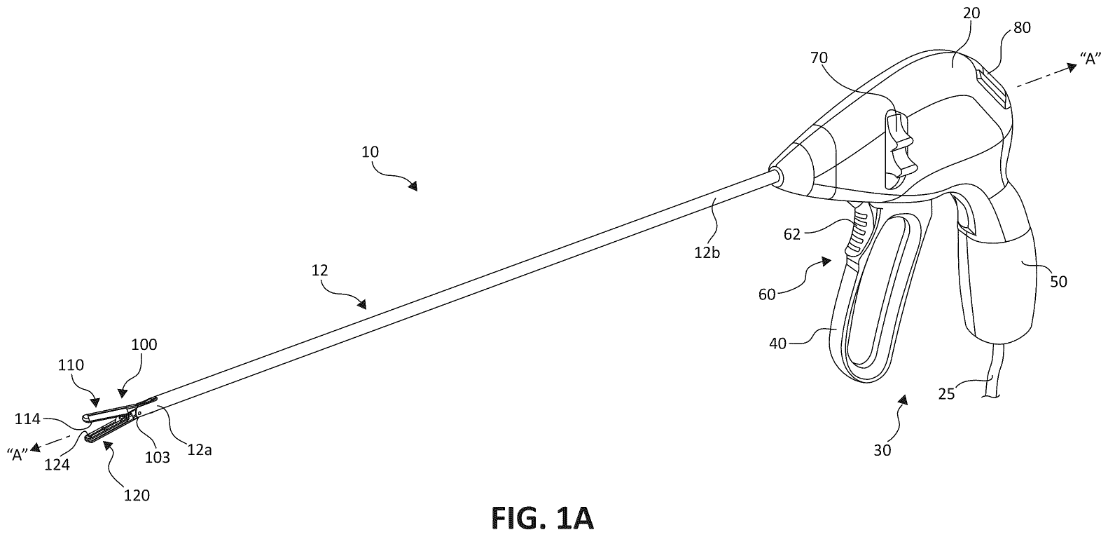

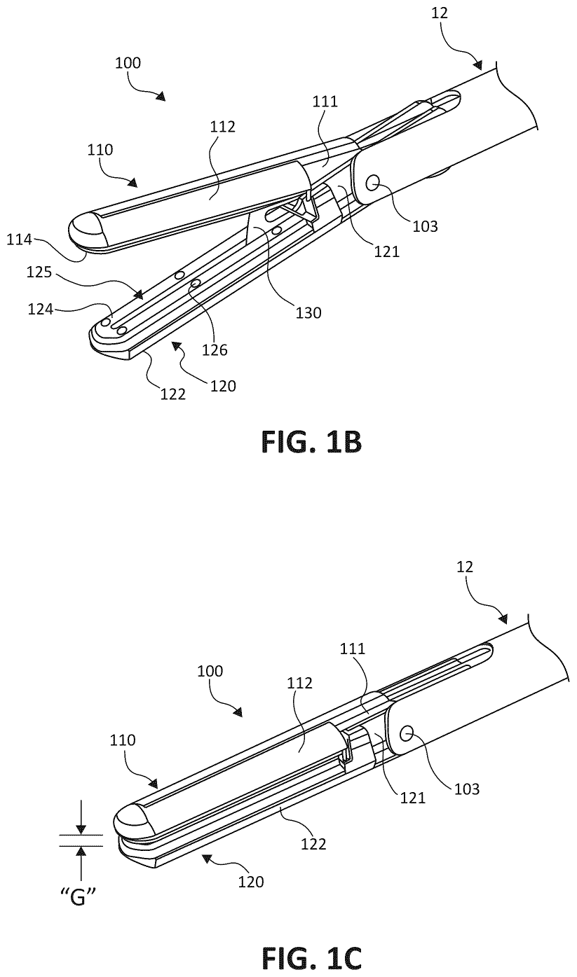

Referring generally to FIG. 1A, an endoscopic surgical forceps exemplifying the aspects and features of the present disclosure is shown generally identified by reference numeral 10. For the purposes herein, endoscopic surgical forceps 10 is generally described. Aspects and features of endoscopic surgical forceps 10 not germane to the understanding of the present disclosure are omitted to avoid obscuring the aspects and features of the present disclosure in unnecessary detail.

Forceps 10 includes a housing 20, a handle assembly 30, a trigger assembly 60, a rotating assembly 70, an activation switch 80, and an end effector assembly 100. Forceps 10 further includes a shaft 12 having a distal end 12a configured to mechanically engage the end effector assembly 100 and a proximal end 12b that mechanically engages the housing 20. Forceps 10 also includes a cable 25 that connects forceps 10 to an energy source (not shown), e.g., a generator or other suitable power source, although forceps 10 may alternatively be configured as a battery-powered device. Cable 25 includes one or more wires (not shown) extending therethrough and having sufficient length to extend through the shaft 12 in order to provide energy to one or both tissue-treating plates 114, 124 of the jaw members 110, 120, respectively, of end effector assembly 100. The tissue-treating plates 114, 124 are electrically coupled to the activation switch 80 and the energy source (not shown). Actuation of the activation switch 80 serves to initiate the delivery of energy from the energy source to the tissue-treating plates 114, 124 for treating, e.g., cauterizing, coagulating/desiccating, and/or sealing, tissue.

The rotating assembly 70 is operably coupled to the shaft 12 so as to enable selective rotation of the shaft 12 and, thus, the end effector assembly 100, relative to the housing 20.

The handle assembly 30 includes a fixed handle 50 and a movable handle 40. The fixed handle 50 is integrally associated with the housing 20 and the movable handle 40 is movable relative to the fixed handle 50. The movable handle 40 is operably coupled to a drive assembly (not shown) that, together, mechanically cooperate to impart movement of one or both of the jaw members 110, 120 about a pivot 103 between a spaced-apart position (FIG. 1B) and an approximated position (FIG. 1C) to grasp tissue between the jaw members 110, 120. The movable handle 40 is initially spaced-apart from the fixed handle 50 and, correspondingly, the jaw members 110, 120 are disposed in the spaced-apart position (FIG. 1B). The movable handle 40 is movable from the initial position toward the fixed handle 50 to move the jaw members 110, 120 to the approximated position (FIG. 1C).

The trigger assembly 60 includes a trigger 62 operably coupled to the housing 20 and movable relative thereto between an un-actuated position and an actuated position. The trigger 62 is operably coupled to a knife assembly, various embodiments of which are detailed below, so as to translate the knife assembly longitudinally to cut tissue grasped between the jaw members 110, 120 upon actuation of the trigger 62. As an alternative to a pivoting trigger 62, a slide trigger, push-button, toggle switch, or other suitable actuator may be provided.

Each of the jaw members 110, 120 includes a proximal flange portion 111, 121, an outer insulative jaw housing 112, 122 disposed about the distal portion (not explicitly shown) of each jaw member 110, 120, and a tissue-treating plate 114, 124, respectively. Proximal flange portions 111, 121 are pivotably coupled to one another about the pivot 103 for moving the jaw members 110, 120 between the spaced-apart and approximated positions, although other suitable mechanisms for pivoting the jaw members 110, 120 relative to one another are also contemplated. The distal portions (not explicitly shown) of the jaw members 110, 120 are configured to support the outer insulative jaw housings 112, 122, and the tissue-treating plates 114, 124, respectively.

In the approximated position, a gap distance “G” may be maintained between the tissue-treating plates 114, 124 by a plurality of stop members 126 (FIG. 1B) disposed on one or both of the tissue-treating plates 114, 124. When the jaw members 110, 120 are in the approximated position, the stop members 126 on one of the tissue-treating plates 114 or 124 contacts the opposing tissue-treating plate 114 or 124 to prohibit further approximation of the tissue-treating plates 114, 124. In some embodiments, the gap distance between the tissue-treating plates 114, 124 when the jaw members 110, 120 are in the approximated position is between about 0.001 inches to about 0.010 inches and, in other embodiments, between about 0.002 and about 0.005 inches. In some embodiments, the stop members 126 are constructed of an electrically non-conductive plastic and molded onto the tissue-treating plates 114, 124, e.g., by a process such as overmolding or injection molding. In other embodiments, the stop members 126 are constructed of a heat-resistant ceramic and deposited onto the tissue-treating plates 114, 124.

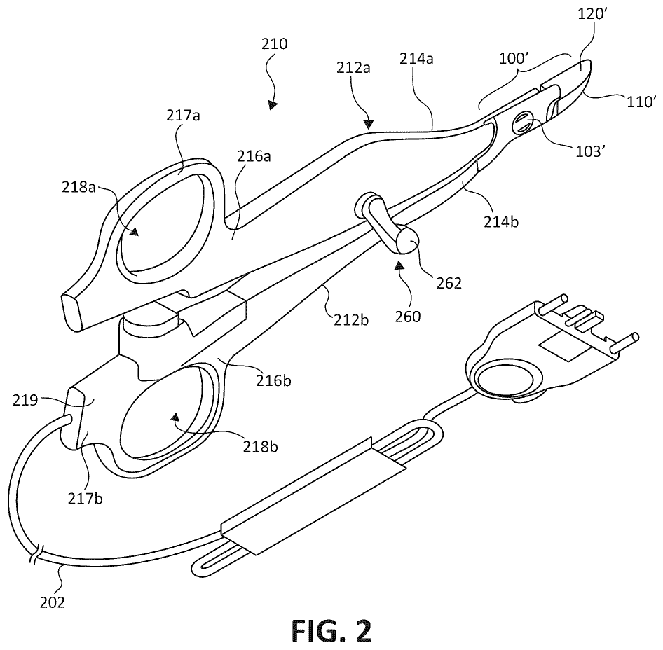

The outer insulative jaw housings 112, 122 of the jaw members 110, 120 support and retain the tissue-treating plates 114, 124 on respective jaw members 110, 120 in opposed relation relative to one another. The tissue-treating plates 114, 124 are formed from an electrically conductive material, e.g., for conducting electrical energy therebetween for treating tissue, although the tissue-treating plates 114, 124 may alternatively be configured to conduct any suitable energy, e.g., thermal, microwave, light, ultrasonic, etc., through tissue grasped therebetween for energy-based tissue treatment. As mentioned above, the tissue-treating plates 114, 124 are electrically coupled to the activation switch 80 and the energy source (not shown), e.g., via the one or more wires (not shown) extending through cable 25 to forceps 10, such that energy may be delivered to the tissue-treating plate 114 and/or the tissue-treating plate 124 and conducted therebetween and through tissue disposed between the jaw members 110, 120 to treat tissue. Once the tissue is treated, a knife blade 130 may be advanced through a longitudinally-extending knife channel 125 defined by one or both of the jaw members 110, 120 (only the knife channel of jaw member 120 is shown in FIG. 2A).

A more detailed description of an endoscopic surgical forceps can be found in commonly owned U.S. Pat. No. 9,820,765, the entire contents of which are incorporated herein by reference.

Referring to FIG. 2, an open surgical forceps exemplifying the aspects and features of the present disclosure is shown generally identified by reference numeral 210. For the purposes herein, open surgical forceps 210 is generally described. Aspects and features of open surgical forceps 210 not germane to the understanding of the present disclosure are omitted to avoid obscuring the aspects and features of the present disclosure in unnecessary detail.

Forceps 210 includes two elongated shaft members 212a, 212b, each having a proximal end 216a, 216b, and a distal end 214a, 214b, respectively. Forceps 210 is configured for use with an end effector assembly 100′ similar to end effector assembly 100 (FIGS. 1 and 2A-2B). More specifically, end effector assembly 100′ includes first and second jaw members 110′, 120′ attached to respective distal ends 214a, 214b of the shaft members 212a, 212b. The jaw members 110′, 120′ are pivotably connected about a pivot 103′. Each shaft member 212a, 212b includes a handle 217a, 217b disposed at the proximal end 216a, 216b thereof. Each handle 217a, 217b defines a finger hole 218a, 218b therethrough for receiving a finger of the user. As can be appreciated, the finger holes 218a, 218b facilitate movement of the shaft members 212a, 212b relative to one another to, in turn, pivot the jaw members 110′, 120′ from the spaced-apart position, wherein jaw members 110′, 120′ are disposed in spaced relation relative to one another, to the approximated position, wherein the jaw members 110′, 120′ cooperate to grasp tissue therebetween.

One of the shaft members 212a, 212b of forceps 210, e.g., shaft member 212b, includes a proximal shaft connector 219 configured to connect forceps 210 to an energy source (not shown), e.g., a generator. The proximal shaft connector 219 secures a cable 202 to forceps 210 such that the user may selectively supply energy to the jaw members 110′, 120′ for treating tissue and for energy-based tissue cutting. More specifically, an activation switch 204 is provided for supplying energy to the jaw members 110′, 120′ to treat tissue upon sufficient approximation of the shaft members 212a, 212b, e.g., upon activation of the activation switch 204 via shaft member 212a.

Forceps 210 further includes a trigger assembly 260 including a trigger 262 coupled to one of the shaft members, e.g., shaft member 212a, and movable relative thereto between an un-actuated position and an actuated position. The trigger 262 is operably coupled to a knife assembly, various embodiments of which are detailed below, so as to actuate the knife assembly to cut tissue grasped between jaw members 110′, 120′ of end effector assembly 100′ upon movement of the trigger 262 to the actuated position. Similarly as noted above, other suitable actuators for the knife assembly are also contemplated.

A more detailed description of an open surgical forceps can be found in commonly owned U.S. patent application Ser. No. 15/593,672 filed on May 12, 2017, the entire contents of which are incorporated herein by reference.

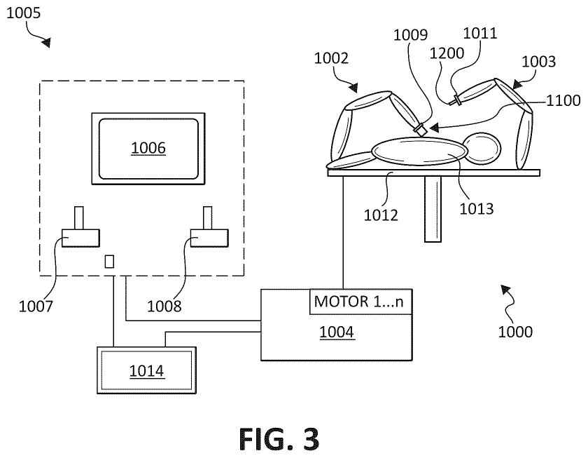

Referring generally to FIG. 3, a robotic surgical system exemplifying the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 includes a plurality of robot arms 1002, 1003; a control device 1004; and an operating console 1005 coupled with the control device 1004. The operating console 1005 may include a display device 1006, which may be set up in particular to display three-dimensional images; and manual input devices 1007, 1008, by means of which a surgeon may be able to telemanipulate the robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to the control device 1004, in which are stored, for example, pre-operative data from the patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to end effector assemblies 100 (FIGS. 1 and 2A-2B) and 100′ (FIG. 3), although other suitable end effector assemblies for coupling to the attaching device 1009 are also contemplated. End effector assembly 1200 may be any end effector assembly, e.g., an endoscopic camera, other surgical tool, etc. The robot arms 1002, 1003 and end effector assemblies 1100, 1200 may be driven by electric drives, e.g., motors, that are connected to the control device 1004. The control device 1004 (e.g., a computer) may be configured to activate the motors, in particular by means of a computer program, in such a way that the robot arms 1002, 1003, their attaching devices 1009, 1011, and end effector assemblies 1100, 1200 execute a desired movement and/or function according to a corresponding input from the manual input devices 1007, 1008, respectively. The control device 1004 may also be configured in such a way that it regulates the movement of the robot arms 1002, 1003 and/or of the motors.

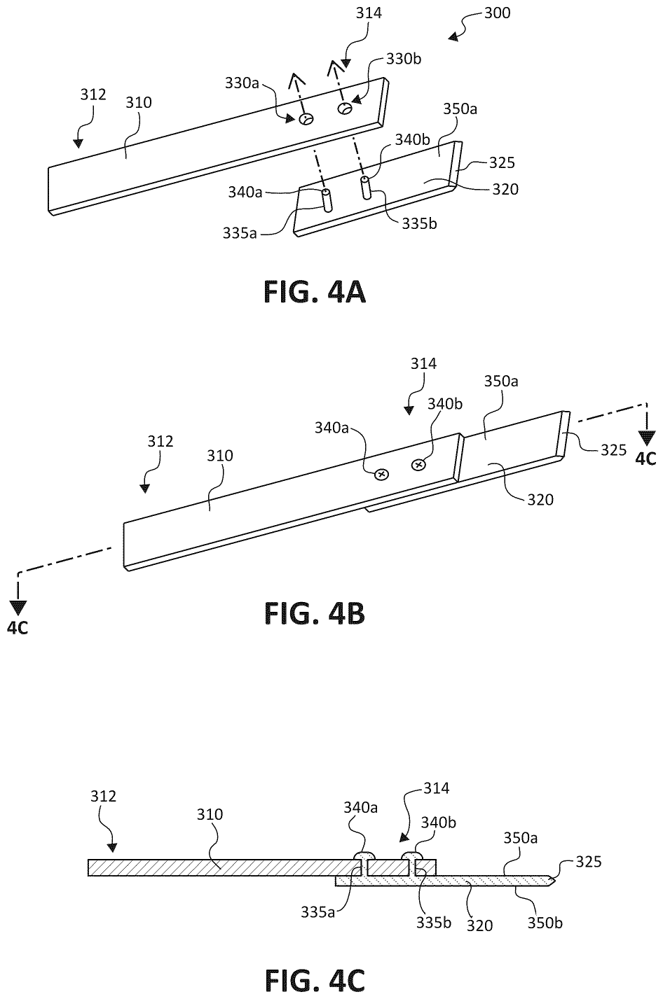

Referring generally to FIGS. 4A-7C, as can be appreciated, challenges are presented in designing knife assemblies for use with surgical instruments having tissue grasping jaws, e.g., forceps 10 (FIGS. 1A-1C), forceps 210 (FIG. 2), and/or robotic surgical system 1000 (FIG. 3). Typically, knife assemblies include an elongated knife having a proximal rod portion that is coupled to a knife actuator (e.g., trigger 62) and a distal portion that includes a sharpened distal edge for cutting tissue. Knife assemblies are typically constructed of a low-cost metal such as stainless steel. However, stainless steel at some grades of hardness may be prone to deformation when advanced through jaws grasping tissue. For example, a distal portion of the knife assembly is configured to advance through a knife channel (e.g., longitudinally-extending knife channel 125) defined through at least one of the grasping jaws to cut tissue grasped by the jaws and then retract from the knife channel after the tissue is cut. If the knife assembly undergoes deformation during advancement through the grasping jaws, the knife assembly may fail to retract from the knife channel of the grasping jaws. To prevent deformation, a super-clastic alloy such as Nitinol may be used to form at least the portion of the knife assembly (e.g., the knife blade) that advances and retracts through the knife channel. However, challenges may be presented when using conventional methods such as welding to couple the super-clastic alloy knife blade component to an elongated shaft component (e.g., rod, wire, cable) that is formed from a different material such as stainless steel. For example, welding a Nitinol knife blade with a stainless steel elongated shaft (or a stainless steel knife blade with a Nitinol elongated shaft) may result in a weak coupling at the weld since welding Nitinol and stainless steel components together is known to either not be possible or, at best, to result in a weak coupling at the weld. If the coupling between the knife blade and elongated shaft breaks during movement of the knife blade within the assembly, the knife blade may exit the knife channel and/or become stuck between the grasping jaws.

Accordingly, the various embodiments of knife assemblies detailed below with respect to FIGS. 4A-7B include a knife blade component configured to cut tissue and to be coupled to an elongated shaft component. The elongated shaft component is configured to operably couple to a knife actuator (e.g., trigger 62) for effecting longitudinal movement of the elongated shaft component and, thus, corresponding longitudinal movement of the knife blade component. In some embodiments, the elongated shaft component may be formed of a first material (e.g., stainless steel) and the knife blade component may be formed of a second material (e.g., Nitinol) different than the first material. The elongated shaft component is configured to be coupled to the knife blade component in a manner that minimizes both the occurrence of the coupling or couplings between the two components breaking and the occurrence of the knife blade component leaving the knife channel when a break in the coupling or couplings between the two components occurs.

With reference to FIGS. 4A-4C, a knife assembly for cutting tissue is provided in accordance with the present disclosure and configured for use with forceps 10 (FIGS. 1-1C), forceps 210 (FIG. 3), robotic surgical system 1000 (FIG. 3), and/or any other suitable surgical instrument or system is shown generally identified by reference numeral 300. The knife assembly 300 generally includes an elongated shaft 310 and a knife blade 320 having a pair of opposing lateral sides 350a, 350b and a distal sharpened edge 325 for cutting tissue. A distal portion 314 of the elongated shaft 310 is configured to be coupled to the knife blade 320 and a proximal portion 312 of the elongated shaft 310 is configured to be operably coupled to a suitable actuator (e.g., trigger 62, trigger 262) for advancing the knife blade 320 through jaw members (e.g., jaw members 110, 120) to cut tissue grasped therebetween. A pair of raised portions 335a, 335b extend generally orthogonally from one of the pair of opposing lateral sides (e.g., lateral side 350a) of the knife blade 320 and terminate at distal end portions 340a, 340b, respectively. During manufacturing of the knife assembly 300, the raised portions 335a, 335b are received through apertures 330a, 330b defined through the distal portion 314 of the elongated shaft 310. Once the raised portions 335a, 335b are received through one end of the respective apertures 330a, 330b of the elongated shaft 310 such that the distal end portions 340a, 340b extend beyond the opposing end of the respective apertures 330a, 330b, the distal end portions 340a, 340b are melted using a suitable heating method (e.g., laser, plasma arc welding, gas tungsten arc welding, or the like) such that the distal end portions 340a, 340b expand to form a generally ball-like shape that has a larger diameter than the rest of the raised portions 335a, 335b, respectively. With this purpose in mind, the raised portions 335a, 335b may be formed from a material (e.g., Nitinol) suitable for melting and expanding in response to the heating process. Once a suitable diameter of the expanded distal end portions 340a, 340b is achieved through the heating process, the expanded distal end portions 340a, 340b are allowed to cool, resulting in the hardening of the expanded distal end portions 340a, 340b. The diameter of the resulting expanded distal end portions 340a, 340b is greater than that of the apertures 330a, 330b through which the raised portions 335a, 335b were received, thereby preventing the raised portions 335a, 335b from retracting out of the respective apertures 330a, 330b. In this manner, the raised portions 335a, 335b are secured within the respective 330a, 330b, respectively, thereby coupling the knife blade 320 to the elongated shaft 310.

In some embodiments, the elongated shaft 310 is formed of a first material (e.g., stainless steel) and the knife blade 320, including the raised portions 335a, 335b, is formed of a second material (e.g., Nitinol) different than the first material.

In some embodiments, the raised portions 335a, 335b may be formed into at least one of the pair of opposing lateral surfaces 350a, 350b of the knife blade 320 using a suitable process such as, for example, a chemical etching process, a laser ablation process, an additive metal process, or a mechanical machining process. In some embodiments, the knife blade 320, including the sharpened distal edge 325 and the raised portions 335a, 335b, is formed using a suitable process such as, for example, a chemical etching process, a laser ablation process, an additive metal process, or a mechanical machining process.

In some embodiments, the raised portions 335a, 335b are formed of Nitinol and are welded to at least one of the pair of opposing surfaces 350a, 350b of the knife blade 320, which is also formed of Nitinol. For example, the raised portions 335a, 335b may be Nitinol wires, separate from the knife blade 320, that are welded to at least one of the pair of opposing lateral sides 350a, 350b of the knife blade 320 such that the Nitinol wires extend generally orthogonally therefrom.

As can be appreciated, the above-noted description of the embodiment of FIGS. 4A and 4B having a pair of raised portions 335a, 335b and a corresponding pair of apertures 330a, 330b is presented for illustrative purposes only and should not be construed as limiting in that any number of raised portions and corresponding number of apertures may be employed for purposes of coupling the elongated shaft 310 to the knife blade 320. For example, a configuration utilizing a single raised portion and a single aperture may be employed or a configuration utilizing any number of a plurality of raised portions and corresponding number of apertures may be employed. Additionally, in some embodiments, the raised portion/aperture configuration may be reversed in that one or more raised portions may extend from the distal portion 314 of the elongated shaft 310 and be received through a corresponding aperture defined through the knife blade 320. Additionally, in some embodiments, the distal portion 314 of the elongated shaft 310 may include any number of both apertures and raised portions that correspond to raised portions and apertures, respectively, of the knife blade 320 for purposes of coupling the elongated shaft 310 to the knife blade 320.

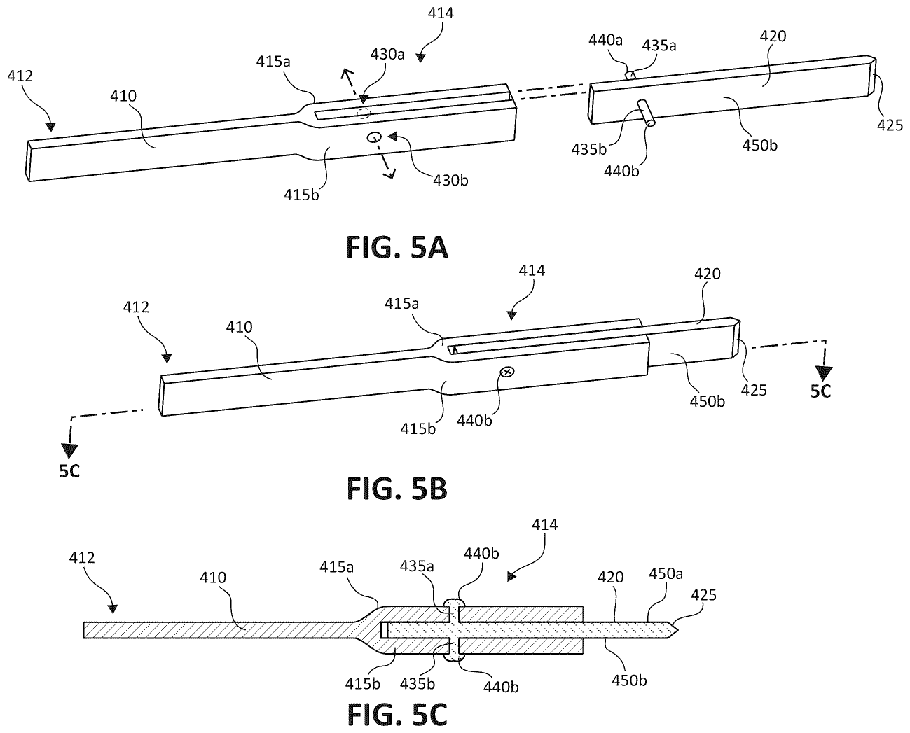

With reference to FIGS. 5A and 5B, a knife assembly for cutting tissue is provided in accordance with the present disclosure and configured for use with forceps 10 (FIGS. 1A-1C), forceps 210 (FIG. 2), robotic surgical system 1000 (FIG. 3), and/or any other suitable surgical instrument or system is shown generally identified by reference numerals 400. The knife assembly 400 generally includes an elongated shaft 410 and a knife blade 420 having a pair of opposing lateral sides 450a, 450b and a distal sharpened edge 425 for cutting tissue. A proximal portion 412 of the elongated shaft 410 is configured to be operably coupled to a suitable actuator (e.g., trigger 62, trigger 262) for advancing the knife blade 420 through jaw members (e.g., jaw members 110, 120) to cut tissue grasped therebetween. A distal portion 414 of the elongated shaft 410 is bifurcated to form a pair of opposing sidewalls 415a, 415b configured to be coupled to the knife blade 420. Each of the opposing sidewalls 415a, 415b define a respective aperture 430a, 430b therethrough. A pair of raised portions 435a, 435b extend from the pair of opposing lateral sides 450a, 450b, respectively, of the knife blade 420. The pair of raised portions 435a, 435b terminate at respective distal end portions 440a, 440b. During manufacturing of the knife assembly 400, the knife blade 420 is received between the opposing sidewalls 415a, 415b and the raised portions 435a, 435b are received through the apertures 430a, 430b defined through the respective sidewalls 415a, 415b. Once the raised portions 435a, 435b are received through one end of the respective apertures 430a, 430b such that the distal end portions 440a, 440b extend beyond the opposing end of the respective apertures 430a, 430b, the distal end portions 440a, 440b are melted using a suitable heating method (e.g., laser, plasma arc welding, gas tungsten arc welding, or the like) such that the distal end portions 440a, 440b expand to form a generally ball-like shape that has a larger diameter than the rest of the raised portions 435a, 435b, respectively. With this purpose in mind, the raised portions 335a, 335b may be formed from a material (e.g., Nitinol) suitable for melting and expanding in response to the heating process. Once a suitable diameter of the expanded distal end portions 440a, 440b is achieved through the heating process, the expanded distal end portions 440a, 440b are allowed to cool, resulting in the hardening of the expanded distal end portions 440a, 440b. The diameter of the resulting expanded distal end portions 440a, 440b is greater than that of the apertures 430a, 430b through which the raised portions 435a, 435b were received, thereby preventing the raised portions 435a, 435b from retracting out of the respective apertures 430a, 430b. In this manner, the raised portions 435a, 435b are secured within the respective 430a, 430b, respectively, thereby coupling the knife blade 420 to the elongated shaft 410.

In some embodiments, the elongated shaft 410 is formed of a first material (e.g., stainless steel) and the knife blade 420, including the raised portions 435a, 435b, is formed of a second material (e.g., Nitinol) different than the first material.

In some embodiments, the raised portions 435a, 435b may be formed into the respective pair of opposing lateral surfaces 450a, 450b of the knife blade 420 using a suitable process such as, for example, a chemical etching process, a laser ablation process, an additive metal process, or a mechanical machining process. In some embodiments, the knife blade 420, including the sharpened distal edge 425 and the raised portions 435a, 435b, is formed using a suitable process such as, for example, a chemical etching process, a laser ablation process, an additive metal process, or a mechanical machining process.

In some embodiments, the raised portions 435a, 435b are formed of Nitinol and are welded to the respective pair of opposing lateral surfaces 450a, 450b of the knife blade 420, which is also formed of Nitinol. For example, the raised portions 435a, 435b may be Nitinol wires, separate from the knife blade 420, that are welded to the pair of opposing lateral sides 450a, 450b, respectively, of the knife blade 420 such that a Nitinol wire extends generally orthogonally from each of the pair of opposing lateral sides 450a, 450b of the knife blade 420.

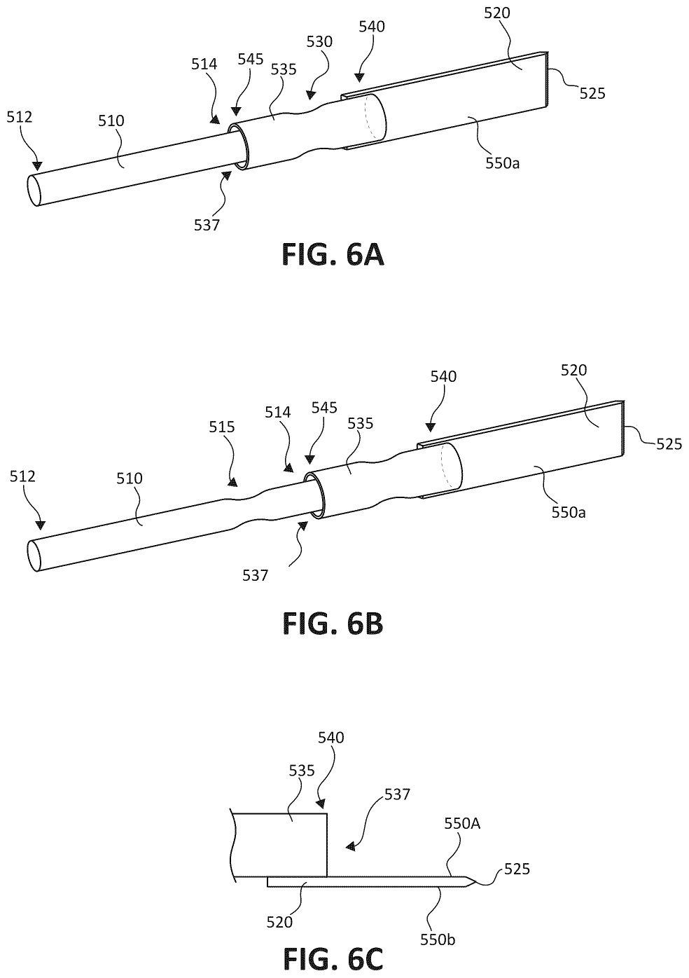

With reference to FIGS. 6A-6C, a knife assembly for cutting tissue is provided in accordance with the present disclosure and configured for use with forceps 10 (FIGS. 1A-1C), forceps 210 (FIG. 2), robotic surgical system 1000 (FIG. 3), and/or any other suitable surgical instrument or system is shown generally identified by reference numerals 500. The knife assembly 500 generally includes an elongated shaft 510, an elongated tube 535 defining a longitudinal lumen 537 therethrough, and a knife blade 520 having a distal sharpened edge 525 for cutting tissue. A proximal portion 512 of the elongated shaft 510 is configured to be operably coupled to a suitable actuator (e.g., trigger 62, trigger 262) for advancing the knife blade 520 through jaw members (e.g., jaw members 110, 120) to cut tissue grasped therebetween. A distal portion 514 of the elongated shaft 510 is configured to be received within the longitudinal lumen 537 from a proximal end portion 545 of the elongated tube 535 for securing the elongated shaft 510 to the elongated tube 535.

In some embodiments, once the elongated shaft 510 is inserted at least partially within the longitudinal lumen 537, the elongated shaft 510 may be welded to the elongated tube 535 in some embodiments. In some embodiments, the portion of the elongated shaft 510 inserted at least partially within the longitudinal lumen 537 may be heated using an energy source (e.g., laser) such that it distorts (e.g., melts) into a shape that applies force against the inner surface of the longitudinal lumen 537 to help maintain the elongated shaft 510 within the longitudinal lumen 537. In some embodiments, molten material from the heated elongated shaft 510 may flow into openings (not shown) formed in the inner surface of the longitudinal lumen 537 to help maintain the elongated shaft 510 within the longitudinal lumen 537. In the scenario where the elongated shaft 510 is formed of Nitinol and the elongated tube 535 is formed of stainless steel, the heated Nitinol elongated shaft 510 does not fuse to the stainless steel elongated tube 535, thereby minimizing the creation of intermetallics that may weaken the elongated shaft 510.

In some embodiments, the knife blade 520 and elongated tube 535 are formed of a first material (e.g., stainless steel) and the elongated shaft 510 is formed of a second material (e.g., Nitinol) different than the first material. As shown in FIG. 6C, the knife blade 520 is configured to be coupled to a distal end portion 540 of the elongated tube 535. In some embodiments, the knife blade 520 may be coupled to an outer surface of the distal end portion 540 of the elongated tube 535 utilizing a suitable method including, but not limited to, welding.

In some embodiments, a portion of the knife assembly 500 may include a reduced profile to provide a weak link having a maximum design strength that is less than the strength of the coupling or couplings between the knife blade 520 and the elongated shaft 510. In this way, if a break or separation of components of the knife assembly 500 is to occur, the weak link will fail before the relatively stronger coupling between the knife blade 520 and the elongated tube 535 fails. For example, in some embodiments the elongated tube 535 may include a reduced profile portion 530, as shown by way of example in FIG. 6A. The reduced profile portion 530 is weaker than the coupling between the elongated shaft 510 and the elongated tube 535 and weaker than the coupling between the elongated tube 535 and the knife blade 520. Thus, if a break or separation of components of the knife assembly 500 is to occur, the elongated tube 535 will fail at the reduced profile portion 530 before the coupling between the elongated tube 535 and the knife blade 520 fails, thereby ensuring that at least a portion of the elongated tube 535 remains coupled to the knife blade 520. As a result, the knife blade 520 is less likely to exit the knife channel (e.g., longitudinally-extending knife channel 125) in this scenario since the elongated tube 535 remains within the surgical instrument (e.g., within the shaft 12 of forceps 10) when the knife blade 520 is advanced and retracted through the grasping jaws via the knife channel.

In some embodiments, the elongated shaft 510 may include a reduced profile portion 515, as shown by way of example in FIG. 6B. The reduced profile portion 515 is weaker than the coupling between the elongated shaft 510 and the elongated tube 535 and weaker than the coupling between the elongated tube 535 and the knife blade 520. Thus, if a break or separation of components of the knife assembly 500 is to occur, the elongated shaft 510 will fail at the reduced profile portion 515 before the coupling between the elongated tube 535 and the knife blade 520 fails, thereby ensuring that the elongated tube 535 and at least a portion of the elongated shaft remains coupled to the knife blade 520. As a result, the knife blade 520 is less likely to leave the knife channel in this scenario since the elongated shaft 510 and the elongated tube 535 remain within the surgical instrument (e.g., within the shaft 12 of forceps 10) when the knife blade 520 is advanced and retracted through the grasping jaws via the knife channel.

During manufacturing of the knife assembly 500, the reduced profile portions 515, 530 may be formed into the elongated shaft 510 and elongated tube 535, respectively, by any suitable method including, but not limited to, chemical etching or tube cutting. In some embodiments, the knife assembly 500 may include reduced profile portion 515 and reduced profile portion 530.

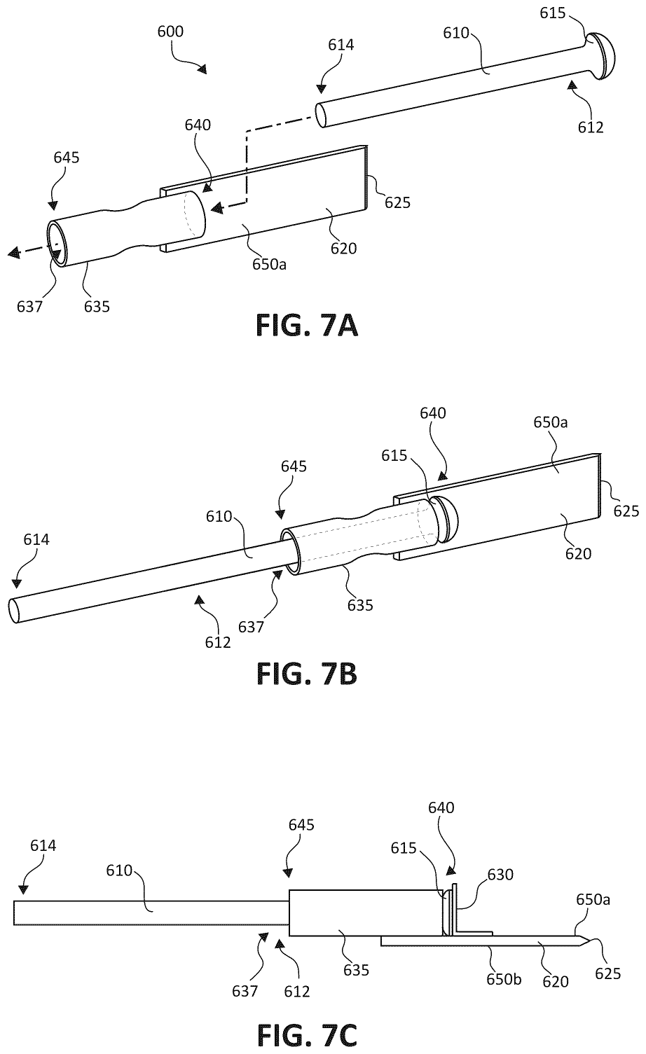

With reference to FIGS. 7A-7C, a knife assembly for cutting tissue is provided in accordance with the present disclosure and configured for use with forceps 10 (FIGS. 1A-1C), forceps 210 (FIG. 2), robotic surgical system 1000 (FIG. 3), and/or any other suitable surgical instrument or system is shown generally identified by reference numerals 600. The knife assembly 600 generally includes an elongated shaft 610 having an enlarged distal end portion 615, an elongated tube 635 defining a longitudinal lumen 637 therethrough, and a knife blade 620 having a pair of opposing lateral sides 650a, 650b and a distal sharpened edge 625 for cutting tissue. A proximal portion 612 of the elongated shaft 610 is configured to be operably coupled to a suitable actuator (e.g., trigger 62, trigger 262) for advancing the knife blade 620 through jaw members (e.g., jaw members 110, 120) to cut tissue grasped therebetween. A distal portion 614 of the elongated shaft 610 is configured to be received within a lumen 637 defined by the elongated tube 635 for coupling the elongated shaft 610 to the elongated tube 635 and the knife blade 620. The knife blade 620 is configured to be coupled to an outer surface of the elongated tube 635 by any suitable method including, but not limited to, welding. The enlarged distal portion 615 may be formed by heating

During manufacturing of the knife assembly 600, the elongated shaft 610 may be inserted through the longitudinal lumen 637 from a distal end portion 640 of the elongated tube 635 and moved proximally until the proximal portion 614 exits the longitudinal lumen 637 at a proximal end portion 645 of the elongated tube 635 and the enlarged distal portion 615 abuts the distal end portion 640 of elongated tube 635 such that further proximal movement of the elongated shaft 610 is prevented. In some embodiments, the elongated shaft 610 may be inserted through the longitudinal lumen 637 from either direction and the enlarged distal portion 615 formed on the distal portion 612 of the elongated shaft 610 after insertion of the elongated shaft 610 through the longitudinal lumen 637. For example, the elongated shaft 610 may be inserted through the longitudinal lumen 637 from the proximal end portion 645 of the elongated tube 635 and moved distally until at least a portion of the elongated shaft 610 exits the distal end portion 640 of the elongated tube 635. Once suitably positioned, the enlarged distal portion 615 may be formed. In some embodiments, the enlarged distal portion 615 is formed by melting one end portion of the elongated shaft 610 using a suitable heating method (e.g., laser, plasma arc welding, gas tungsten arc welding, or the like) such that the heated portion of the elongated shaft 610 expands to form a generally ball-like shape of the enlarged distal portion 615, which has a larger diameter than the rest of the elongated shaft 610. With this purpose in mind, the elongated shaft 610 may be formed from a material (e.g., Nitinol) suitable for melting and expanding in response to the heating process. Once a suitable diameter of the enlarged distal portion 615 is achieved through the heating process, the enlarged distal portion 615 is allowed to cool, resulting in the hardening of the enlarged distal portion 615. The diameter of the resulting enlarged distal portion 615 is greater than that of the longitudinal lumen 637 through which the elongated shaft 610 is received, thereby preventing the further proximal movement of the elongated shaft 610 once the enlarged distal portion 615 abuts the distal end portion 640 of the elongated tube 635.

In some embodiments, a shaft stop 630 that serves to prevent distal movement of the elongated shaft 610 relative to the knife blade 620 may be coupled to the knife blade 620 distal to the coupling between the elongated tube 635 and the knife blade 620 and distal to the enlarged distal portion 615, as shown by way of example in FIG. 7C. In some embodiments, the shaft stop 630 may be coupled to one of the pair of opposing lateral sides (e.g., 650a) of the knife blade 520 utilizing a suitable method including, but not limited to, welding.

In some embodiments, the knife blade 620 and the elongated tube 635 may be formed from the same material (e.g., stainless steel) and the elongated shaft 610 may be formed from a material (e.g., Nitinol) different than that of the knife blade 620 and the elongated tube 635.

Typically, crimping is used to couple a Nitinol component to a component of a different material (e.g., stainless steel). In some embodiments, the elongated shaft 610 is formed of Nitinol and the knife blade 620 and elongated tube 635 are formed of stainless steel. In this scenario, once the elongated shaft 610 is received through the longitudinal lumen 637 and the enlarged distal portion 615 abuts the distal end portion 640 of the elongated tube 635, the enlarged distal portion 615 may be crimped to the elongated tube 635. The enlarged distal portion 615 increases the friction of the crimp joint, which enables the crimp to be relatively smaller while maintaining the strength of a larger crimp and reduces the requirements for material thickness and strength of the crimp incident on the elongated shaft 610.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1-20. (canceled)

21. An electrosurgical instrument, comprising:
an end effector configured to grasp tissue;
a tube having a lumen;
an elongated shaft having a distal end portion received within the lumen of the tube, the distal end portion attached to an inner surface of the tube via a first coupling; and
a knife blade having a pair of opposing lateral sides, one of the lateral sides of the knife blade being attached to an outer surface of the tube via a second coupling such that longitudinal movement of the elongated shaft moves the knife blade through the end effector for cutting tissue, wherein the tube is configured to mechanically fail prior to the first and second couplings in response to an applied stress.

22. The electrosurgical instrument according to claim 21, wherein the tube includes a reduced profile portion that is mechanically weaker than the first and second couplings such that the reduced profile portion of the tube is configured to mechanically fail prior to the first and second couplings in response to the applied stress.

23. The electrosurgical instrument according to claim 21, wherein the elongated shaft includes a reduced profile portion that is mechanically weaker than the first and second couplings such that the reduced profile portion of the elongated shaft is configured to break prior to the first and second couplings in response to the applied stress.

24. The electrosurgical instrument according to claim 21, wherein the knife blade and the tube are formed from stainless steel.

25. The electrosurgical instrument according to claim 21, wherein the elongated shaft is formed from Nitinol.

26. The electrosurgical instrument according to claim 21, wherein the knife blade is formed from a material that is different than at least one of a material from which the elongated shaft is formed or a material from which the tube is formed.

27. The electrosurgical instrument according to claim 21, wherein the distal end portion of the elongated shaft is welded to the inner surface of the tube at the first coupling.

28. The electrosurgical instrument according to claim 21, wherein the outer surface of the tube is welded to the one lateral side of the knife blade at the second coupling.

29. The electrosurgical instrument according to claim 21, wherein the electrosurgical instrument is operably coupled to a robotic surgical system.

30. The electrosurgical instrument according to claim 21, wherein the end effector includes a pair of jaw members configured to grasp the tissue, at least one jaw member of the pair of jaw members including a knife channel configured to receive the knife blade.

31. An electrosurgical instrument, comprising:
a pair of jaw members configured to grasp tissue, at least one jaw member of the pair of jaw members including a knife channel;
a tube having a lumen;
an elongated shaft having a distal end portion received within the lumen of the tube, the distal end portion attached to an inner surface of the tube via a first coupling; and
a knife blade having a pair of opposing lateral sides, one of the lateral sides of the knife blade being attached to an outer surface of the tube via a second coupling such that longitudinal movement of the elongated shaft moves the knife blade through the knife channel for cutting tissue, wherein the tube is configured to mechanically fail prior to the first and second couplings in response to an applied stress.

32. The electrosurgical instrument according to claim 31, wherein the tube includes a reduced profile portion that is mechanically weaker than the first and second couplings such that the reduced profile portion of the tube is configured to mechanically fail prior to the first and second couplings in response to the applied stress.

33. The electrosurgical instrument according to claim 31, wherein the elongated shaft includes a reduced profile portion that is mechanically weaker than the first and second couplings such that the reduced profile portion of the elongated shaft is configured to break prior to the first and second couplings in response to the applied stress.

34. The electrosurgical instrument according to claim 31, wherein the knife blade and the tube are formed from stainless steel.

35. The electrosurgical instrument according to claim 31, wherein the elongated shaft is formed from Nitinol.

36. The electrosurgical instrument according to claim 31, wherein the knife blade is formed from a material that is different than at least one of a material from which the elongated shaft is formed or a material from which the tube is formed.

37. The electrosurgical instrument according to claim 31, wherein the distal end portion of the elongated shaft is welded to the inner surface of the tube at the first coupling.

38. The electrosurgical instrument according to claim 31, wherein the outer surface of the tube is welded to the one lateral side of the knife blade at the second coupling.

39. The electrosurgical instrument according to claim 31, wherein the electrosurgical instrument is operably coupled to a robotic surgical system.

40. An electrosurgical instrument, comprising:
a pair of jaw members configured to grasp tissue, at least one jaw member of the pair of jaw members including a knife channel;
a knife blade configured to move within the knife channel to cut the tissue;
an elongated shaft configured to move the knife blade within the knife channel; and
a tube having an inner surface attached to a distal end portion of the elongated shaft via a first coupling and an outer surface attached to the knife blade via a second coupling, wherein the tube is configured to mechanically fail prior to the first and second couplings in response to movement of the knife blade within the knife channel such that the knife blade remains within the knife channel upon mechanical failure of the tube.
